(19) United States
(12) Patent Application Publication
(10) Pub. No.: US 2006/0029683 A1
(43) Pub. Date: Feb. 9, 2006

(54) Appendix of 2.7% ~ 9% saline, 56% saline

(76) Inventor: ARan Kim, Jeonju (KR)

Correspondence Address: ARan Kim, Hyundai Apt 106-1509, Hyoja Dong, Jeonju, Jeonbuk, 560-240, KR

(21) Appl. No.: 11/017,708

(22) Filed: Sep. 5, 2005

(30) Foreign Application Priority Data
Feb. 25, 2004 (KR) 1-1-2004-5031376-

(51) Int. Cl. A61K 33/14

(52) U.S. Cl. 424/680

(57) ABSTRACT

The name of invention is Saline and the Saline is NaCl sol. is distinguished by density. In this invention solution, Pus at wound can be removed without curettage and antibiotics by lysis of phosphatide of H+. In 2.7%˜9% Saline sol. of in potential power of semipermeable memb., Membrane formation and cell mutiplication become by electric passive transport of H+. In this sol. cell culture is possible. In 56%(9%˜56%) Saline sol. out of potential power of semipermeable memb. thick memb. be made, and the thick memb. can substituted for skin. Unnecessary cell can be removed without operation by memb. disfunction of Osmosis. And Transformation of tissue can be repaired by absorption press.

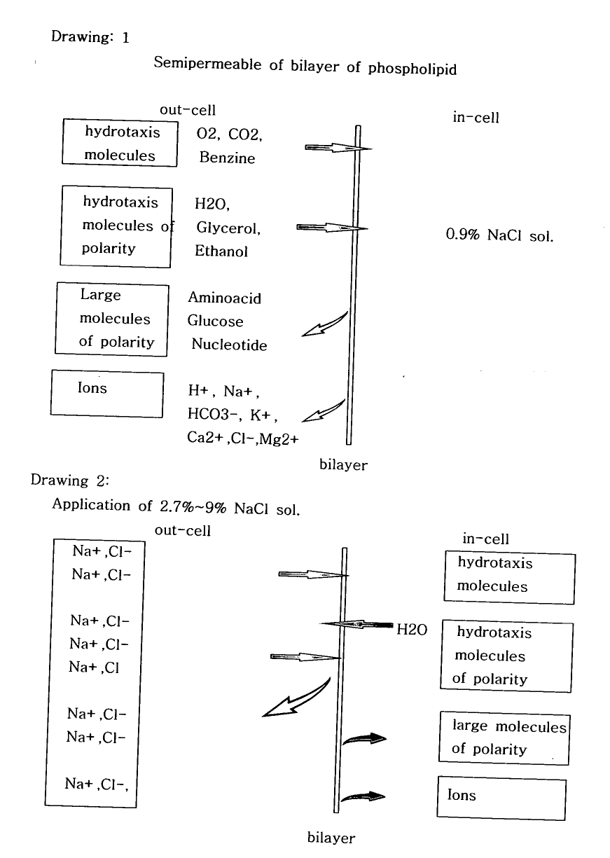

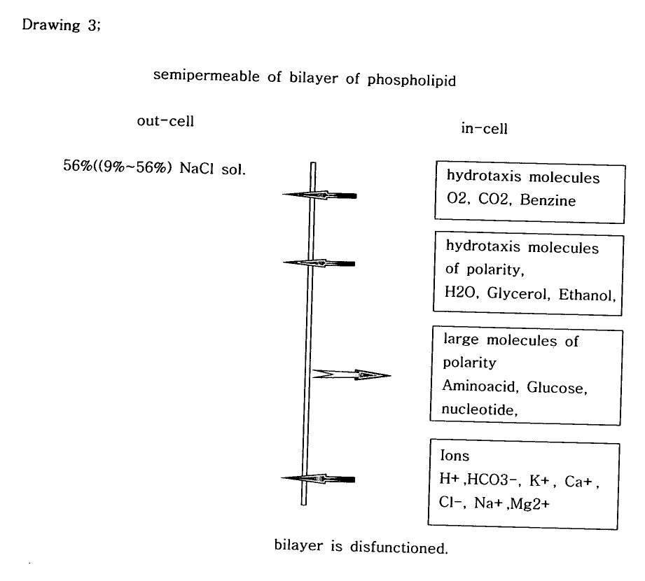

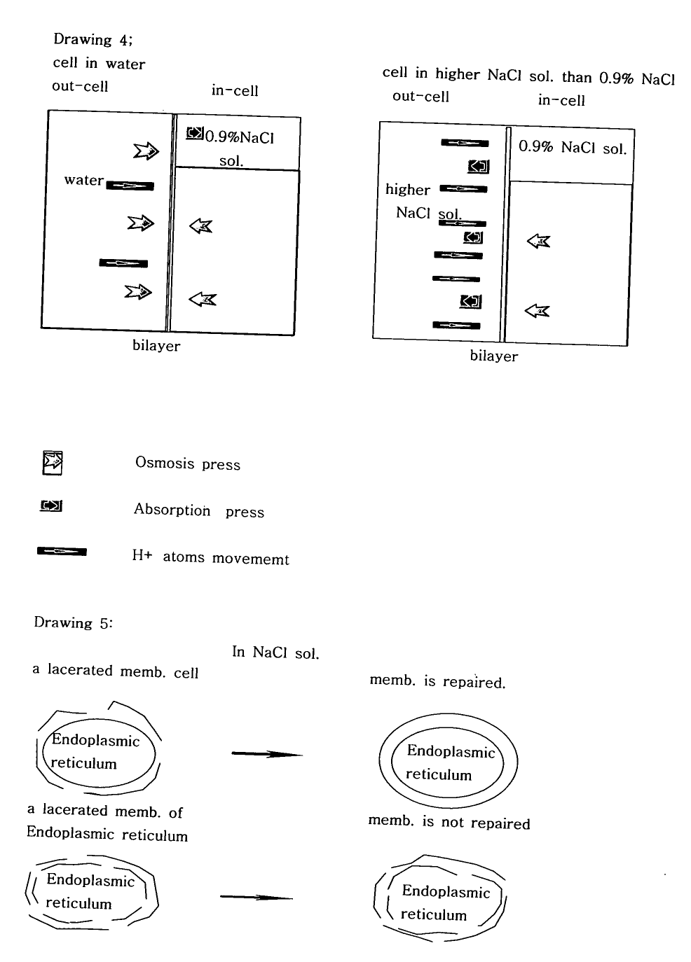

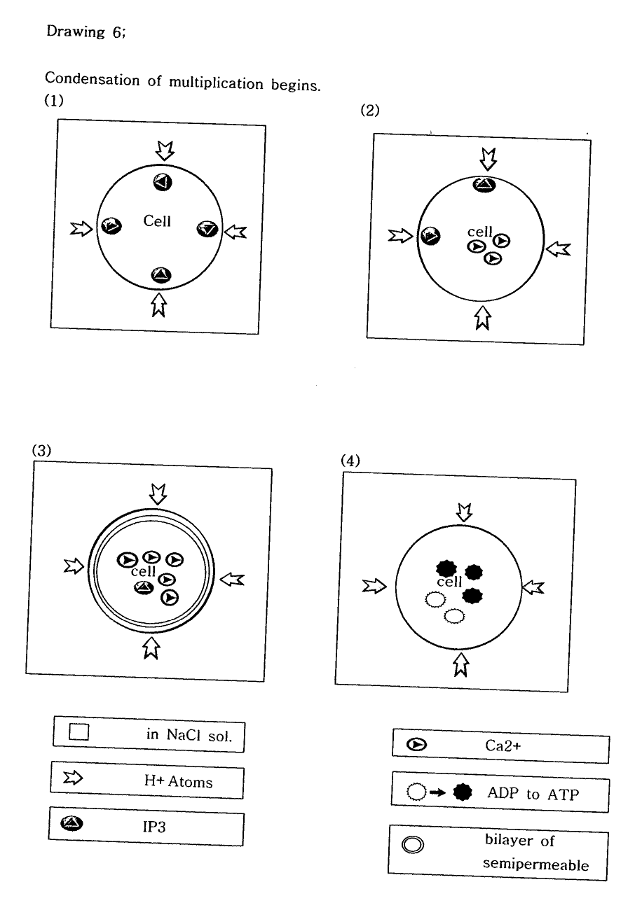

### FIELD OF THE INVENTION

This invention can be used widely in surgery part for pus to remove, membrane to make, unnecessary cell to extract, cell to multiply,

In Gynecology, for bacilli to clean. If research continue, maybe pregnancy period can be shortened by density of amniotic fluid is highly invented. 10 months to 7˜8 months.

In ENT part, symtom of Tonsillitis, Sinusitis can be reduced. Thin cornea can be become thick by irrigation with this sol. near-sightedness can be treated.

56%(9%˜56%) sol. can be used in surgery part for skin to substitute. and it can be used as antiseptic sol. It can be used as strong absorption press.

In dietetics, on aminoacid as yeast.

### BACKGROUND OF THE INVENTION

This Invention is not examinated. It is a reason. So, background of invention only can describe my experience.

As I was a young girl, I accidentally treated Sinusitis by irrigation with salt sol. I noticed the fact that salt sol. can remove pus on wound after long time.

I knew nails of fingers and toes became transformation since hands and feet were exposed in dirty water long time. Nails divided two and three pieces. When I applied salt sol. on the nails, the nails became normal shape.

As I worked in hospital, it was better that Saline (0.9%NaCl) was used than any other sol. was used on dressing wound. The wound seemed like to have more vitality. Cell multiplication was quicker.

About potential power of semipermeable memb. The power is written on klinisches Weorterbuch “3 partition˜10 partition”.

What is the potential power of memb. against?. Memb. take nutritions by the ways “Osmosis, Diffussion and Filtration”

Blood can supply substances by the three ways above to memb. And the density of blood is 0.9% NaCl sol.

So, if one calculate it on the base above, the scope of the potential power is 2.7%˜9% NaCl sol.

### OBJECTS OF THE INVENTION

This Invention has not reflection like antibiotic.

This Invention has not nearly risk. Only If high density sol is used, memb. will be become thick. It is a contraindication.

The salt is economic substance. Expenditure for to treat is cheap.

The Invention has affinity to vitality of cell.

The using is easy.

### SUMMERY OF THE INVENTION

The quality of the Invention is accorded to nature of semipermeable and permeable memb., by electric and passive transport.

Another quality of Invention is accorded to nature of exertion of Osmosis on semipermeable memb., by absorption press.

Each salt package will be parted by gram and the old biosalt sol. 0.9% Saline need for constitution of the Invention. If it is needed 9% Saline, the salt package to 8 gm can be mixed into 0.9% Saline sol. 1000 cc.

### DETAILED DESCRIPTION OF THE INVENTION

This Invention can be used on semipermeable memb. and permeable memb. and divided to two types that the density is in potential power of semipermeable memb., 2.7%-9% NaCl sol. and over potential power, 9%-56% NaCl sol.

NaCl divide to Na+,Cl− in water. It means NaCl in solution has double power. and Na+, and Cl− don't pass semipermeable memb. So, NaCl sol. works on memb. above as absorption press.

In water, atoms exist as each other give and take atoms. In NaCl sol. more atoms than in water have activity. The atoms are NaOH, H+, Cl+H2O.

H+ atoms of them act for lysis of phosphatase, change ATP to ADP. and promote to change ADP to ATP.

By the explain above, H+ atoms cause electric and passive transport in sol.

In NaCl sol. many H+ atoms exist.

When this sol. apply on wound with pus(died WBC and bacilli), pus can be solved by H+ atoms, the lived bacilli, permeable, protect selves as to make memb. be thick and to make cell be hard with Ca+. The same developing occurs at lived semipermeable. The pus become to extract and memb.s be contracted. So, the pus can be extracted from tissue.

In NACl sol. the lacerated memb. can be repaired if Endoplasmic reculum is complete. In higher NaCl sol. memb. can be thickened and it be substituted as skin.

In NaCl sol. transformation of tissue can be repaired. Protein, main component of tissue, is folded by hydrogen bond, ionic bond and van der waals power. Hydrogen bond be weakened by alcohol, metals, ions and it connect to transformation of tissue. The absorption press against osmosis press extract the hydrotaxis molecules and the hydrotaxis molecules of polarity in cell to out-cell. This the factors make hydrogen bond be weak, can be discharged to out cell. The substances can not be solved in water, can not be extracted.

In this sol. cell multiplication and culturing become. But, this sol. has scope as the density in potential power and assemipermeable memb. 2.7%˜9% NaCl sol must be used.

Cell multiplication and culturing depend on electric and passive transfort of H+ atoms in sol.

H+ atoms work on bilayer of phospholipid as phospholipase. IP3 let Ca2+ be active. Ca2+ make cell be condensative. The continued input of H+ atoms come in the condition as to change ADP to ATP. The condition above make cell multiplication and cell culturing be possible. Increasing of H+ atoms means that Enzymes with H+ atomes increase. So, it is importent which substances with H+ atoms are existed in the solution.

The higher NaCl sol. than potential power can be worked on the condition above as contraindication.

### Detailed Description of the Accompanying Drawings

Drawing 1: It is appeared semipermeable of bilayer of phospholipid. hydrotaxis molecules and hydrotaxis molecules of polarity pass the bilayer of phospholipid. And, large molecules of polarity and ions don't pass the bilayer of phospholipid. Osmosis, diffusion and filtration are in normal processing.

Drawing 2: When NaCl sol. in potential power and in higher sol. than biosalt(0.9%) sol. is applied, Osmosis don't occur in out-cell. Absorption press works. And Diffusion and Filtration are possible, if the substance depend upon electric transport and passive transport of H+ atoms.

Drawing 3: When NaCl sol. over potential power, is applied, memb. become disfunction. Osmosis has strong power. Diffusion and filtration have limit in according to the density.

Drawing 4: It is appeared semipermeable memb. in NaCl sol. In NaCl sol. H+ atoms increase so the movement of the atoms increase too. And opposition of Osmosis press, absorption press will be become strong. because of the many H+ atoms, electric passive transport work actively.

Drawing 5: When the memb. of Endoplasmic reticulum is complete, the laceration of memb. of cell can be repaired because memb. can be made in Endoplasmic reticulum.

Drawing 6: (1) It appeared H+ atoms work as phosphatase and phoaspholipase at bilayer of phospholipid. Lysis of phosphatide occurs.

(2) Example, Phospholipase let IP3 cause in cell. IP3 open the memb. of Endoplasmic reticulum and let escape Ca2+.

(3) It show the condensasion of cell.

(4) H+ atoms relate to promote various enzyme. It is appeared H+ atoms are promoting ADP to ATP and ATP to ADP.

This Invention has economic value, affinity for tissue, easily using and small reaction. Using this Invention, even if an operation needs, is main purpose. This Invention don't be used for an operation, the operation applies for the using of this Invention.

I believe this Invention will be used for useful purpose of the world.

What is claimed is:

1. NaCl solution has higher density than Biosalt sol. (0.9%), divide largely to two types in the density. It is named 2.7%˜9% Saline and 56% (9%˜56%) Saline.

2. The density is decided by potential power of semipermeable memb. there are (i) 2.7%˜9% sol. in potential power and (ii) 9%˜56% sol. over potential power.

3. Limit of the potential power of semipermeable memb. is 3 partition˜10 partition. and the potential power is made of against NaCl. If the partitions are changed to percentage, it became 2.7%˜9% NaCl sol.

4. NaCl sol. have (a) higher absorption power of against osmosis power, (b) higher H+ movemrnt, let occur passive transport and (c) larger polarity by H+, let occur electric transport than in normal water. In the sol. NaOH, H+, H2O, Cl atoms be moving busily.

5. Bilayer of semipermeable memb. transmit hydrotaxis molecules (Co2, O2, Benzine) and hydrotaxis molecules of polarity (H2O, Glycerol, Ethanol).

6. Bilayer of semipermeable memb. don't transmit large molecules of polarity (Aminoacid, Glucose, Necleotide) and Ions (H+ Na+ HCo3−, K+, Ca2+, Cl−.Mg2+)

7. When 2.7%-9% NaCl sol. is used on out-cell, claim 4(a), wherein osmosis press don't occur in out-cell and absortioption press works. But, diffusion and filtration depend upon electric transport and passive transport of H+ atoms, occur.

8. By claim 4(b), H+ atoms, in according to be vitality of cell or non-vitality cell, can be differed effect on phophatide.
H+ atoms let occur lysis of phophatide, that is
ATP change to ADP by H+ atoms. ADP be promoted to ATP with energy, in many H+ atoms condition.

9. At non-vitality cell, Pus (died WBC) can be solved by high H+ atoms, because of lysis of phosphatide. ATP change to ADP.
There is limit for ADP to change to ATP, because of non-vitality cell. pus be removed from the clipped cell.

10. At vitality cell, when high H+ atoms exist at phospholipid of Bilayer, the cell has the ability that change ADP to ATP, because energy can be supplied.

11. claim 9 use on Bacilli, vitality cell. when H+ atoms come in to cell of bacilli, Ca2+ in bacilli appears by H+ atoms and bacilli cell be contracted by action of Ca2+.

12. Virus can be killed because multiplication of virus depended on Plasmid. It seems like Plasmid need, because Virus cannot let appear Ca2+.

13. claim 2(ii), 9%˜56% NaCl sol. bring disfunction of memb. hydrotaxis molecules, hydrotaxis molecules of polarity and ions come out of cell, and large molecules of polarity leave in cell as corn by claim 4(a). The density, corn can be made, can be differed by classes and the during.

14. When this sol. is applied on unnecessary cell, by claim 12, unnecessary cell becomes corn and come out from tissue.

15. When this sol. is applied on open wound, memb. become more and more thicker, according to increase density, it can be substituted skin.

16. On bacilli when this high density sol. is applied, can be become antiseptic sol.

17. It can be used on open wound of AISD without pus.

18. Protein, main component of tissue, is folded to hydrogen bond, ionic bond and van der waals power.

19. This three bonds can be folded in water, because Atoms move active in water.

20. If alcohol, ions, heavy metals connect with hydrogen bond, they make hydrogen bond be weak.

21. The weakening of hydrogen bond brings transformation of tissue.

22. In claim 2(i), by claim 4(a), the factors make hydrogen bond be weak, can be discharged out of cell.

23. and then the transformation will be repaired.

24. In vitality tissue, if the condition that fluid can be come in and out freely is, NaCl sol. must be used high density for transformation to repair

25. There are factors too can be not removed with NaCl solution. It must be given careful care on the substances do not solve in water.

26. If memb. of cell is lacerated and challenged against potential power, if the memb. of endoplasmic reticulum is complete, the lacerated memb. of cell can be repaired, because memb. be made in endoplasmic reticulum, and the memb., is challenged, becomes thicker and thicker by density.

27. NaCl sol. of lower density than high limit of potential power, will be used for multiplication of cell.

28. Cell function for energy formation and cell multiplication.

29. Energe formation require water.

30. Cell multiplication require H+ atoms in water. and polarity molecules in water can be changed to acid and base.

31. So, cell mutiplication is possible in water, but in water, because H2O come into cell by Osmosis, the multiplication can be concluded in according to change ADP to ATP. There is limit on the processing in normal water.

32. By claim 4(a)(b)(c), in NaCl sol. cells and neuro cells multiplication and cell culturing occur.

33. This sol. gives more vitality to cell by claim 4(b)(c).

34. At bilayer of phospholipid, when G-protein make phospholipase C be active, phospholipase C let hydrolysis do at the memb.

35. And then IP3 will be made of phospholipid. the IP3 in cell opens the path of Ca2+ and Ca2+ escape from endoplamic reticulum.

36. Diffusion of Ca2+ shows a sign of cell mutiplication that cell become condensation.

37. In NaCl sol, increasing of H+ atoms make actively ATP change to ADP, ADP to ATP in vitality cell by electric passive transport of H+ atoms.

38. The action of H+ above charge for multiplication and culturing of cell.
